(19) United States
(12) Patent Application Publication
(10) Pub. No.: US 2026/0049983 A1
(43) Pub. Date: Feb. 19, 2026

(54) RAPID TEST SYSTEM FOR DETECTING FENTANYL CONCENTRATION

(71) Applicant: Quanta Vision Co., Ltd., New Taipei City (TW)

(72) Inventors: CHIH-YI HSU, New Taipei City (TW); YI-SHAN WENG, New Taipei City (TW); YA-CHIEH YU, New Taipei City (TW)

(21) Appl. No.: 19/298,629

(22) Filed: Aug. 13, 2025

(30) Foreign Application Priority Data
Aug. 16, 2024 (TW) 113130926

(51) Int. Cl. G01N 33/543; G01N 33/94

(52) U.S. Cl. CPC G01N 33/54387; G01N 33/9486

(57) ABSTRACT

A rapid test system for detecting fentanyl concentration is provided, the rapid test system includes a base layer, a sample pad, a first conjugate pad, a second conjugate pad, a reaction pad and an absorption pad, the sample pad, the first conjugate pad, the second conjugate pad, the reaction pad and the absorption pad are sequentially arranged above the base layer along a first direction, the first conjugate pad includes a plurality of gold conjugated fentanyl antibody complexes, the second conjugate pad includes a plurality of gold conjugated trithiocyanuric acid complexes, and the plurality of gold conjugated fentanyl antibody complexes are configured to bind with fentanyl.

### BACKGROUND OF THE INVENTION

### Field of the Invention

The present invention relates to a rapid test system, particularly to a rapid test system for detecting fentanyl concentration.

### Description of the Prior Art

Fentanyl is a synthetic opioid drug, commonly used for treating severe pain, such as cancer pain or postoperative pain. However, in addition to pain relief, it is highly addictive and can cause both physiological and psychological dependence, which can develop into drug abuse and harm health.

In Taiwan, fentanyl is classified as a schedule II controlled drug and is strictly regulated to prevent misuse. Currently, there are many methods for detecting fentanyl, such as using fentanyl urine test strips, fentanyl powder test strips, and fentanyl saliva test strips. However, the fentanyl detection threshold of the above test strips is 20 ng/mL, which results in an inability to detect lower concentrations of fentanyl and leads to the disadvantages of low detection sensitivity and weak detection signal.

The present invention is, therefore, arisen to obviate or at least mitigate the above-mentioned disadvantages.

### SUMMARY OF THE INVENTION

The main object of the present invention is to provide a rapid test system for detecting fentanyl concentration that is capable of detecting fentanyl in saliva and provides a high-intensity detection signal.

To achieve the above and other objects, a rapid test system for detecting fentanyl concentration is provided, the rapid test system includes a base layer, a sample pad, a first conjugate pad, a second conjugate pad, a reaction pad and an absorption pad, the sample pad, the first conjugate pad, the second conjugate pad, the reaction pad and the absorption pad are sequentially arranged above the base layer along a first direction, the first conjugate pad includes a plurality of gold conjugated fentanyl antibody complexes, the second conjugate pad includes a plurality of gold conjugated trithiocyanuric acid complexes, and the plurality of gold conjugated fentanyl antibody complexes are configured to bind with fentanyl.

The present invention will become more obvious from the following description when taken in connection with the accompanying drawings, which show, for purpose of illustrations only, the preferred embodiment(s) in accordance with the present invention.

### BRIEF DESCRIPTION OF THE DRAWINGS

FIG. 1 is a schematic structural diagram of an exemplary embodiment of the present invention;

FIG. 2 is a top view schematic structural diagram of an exemplary embodiment of the present invention;

FIG. 3 is a diagram showing a usage state without fentanyl in the specimen of an exemplary embodiment of the present invention;

FIG. 4 is a diagram showing a usage state with fentanyl in the specimen of an exemplary embodiment of the present invention; and

FIG. 5 includes an experimental result diagram of the specimen and a graph showing the relationship between specimen concentration and difference value of the present invention.

### DETAILED DESCRIPTION OF THE PREFERRED EMBODIMENTS

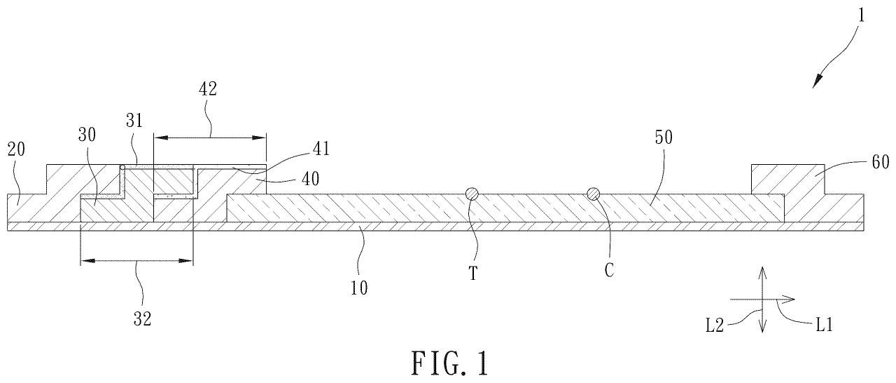

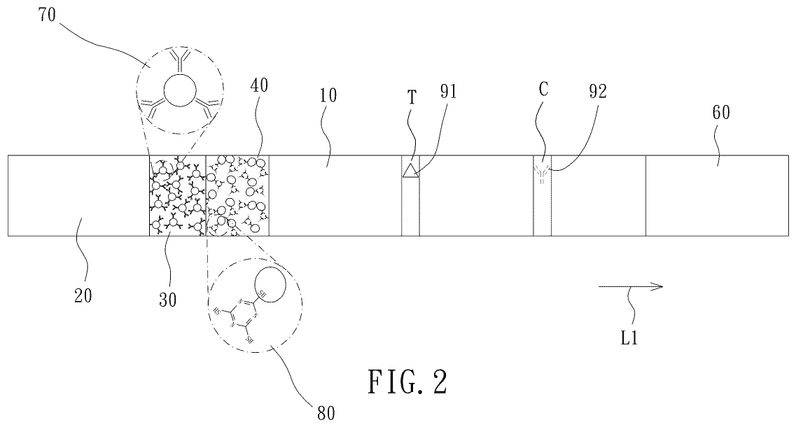

Please refer to FIGS. 1 to 5 for an exemplary embodiment of the present invention. A rapid test system for detecting fentanyl concentration of the present invention includes a base layer 10, a sample pad 20, a first conjugate pad 30, a second conjugate pad 40, a reaction pad 50, and an absorption pad 60.

The sample pad 20, the first conjugate pad 30, the second conjugate pad 40, the reaction pad 50 and the absorption pad 60 are sequentially arranged above the base layer 10 along a first direction L1. The first conjugate pad 30 includes a plurality of gold conjugated fentanyl antibody complexes 70. The second conjugate pad 40 includes a plurality of gold conjugated trithiocyanuric acid complexes 80. The plurality of gold conjugated fentanyl antibody complexes 70 are configured to bind with fentanyl 3. Thereby, fentanyl 3 in a specimen 2, 2a (e.g., saliva) can be detected, and the system provides high-intensity detection signal, high detection sensitivity, and rapid detection.

Each of the plurality of gold conjugated trithiocyanuric acid complexes 80 is formed by trithiocyanuric acid 82 being bound to conjugate gold 81 via a gold-sulfur bond.

The base layer 10 may be a polyvinyl chloride (PVC) sheet or a sheet of other material.

The plurality of gold conjugated fentanyl antibody complexes 70 are disposed by spraying to form a first coating layer 31 covering the first conjugate pad 30. The plurality of gold conjugated trithiocyanuric acid complexes 80 are formed by spraying and disposed over a second coating layer 41 of the second conjugate pad 40. Preferably, an amount of a sprayed liquid of the plurality of gold conjugated fentanyl antibody complexes 70 on the first conjugate pad 30 is equal to an amount of a sprayed liquid of the plurality of gold conjugated trithiocyanuric acid complexes 80 on the second conjugate pad 40. Specifically, the amount of the sprayed liquid of the plurality of gold conjugated fentanyl antibody complexes 70 on the first conjugate pad 30 and the amount of the sprayed liquid of the plurality of gold conjugated trithiocyanuric acid complexes 80 on the second conjugate pad 40 are both 5.00±1.00 μL. Thus, high accuracy, high detection sensitivity, and high signal intensity are achieved.

In this embodiment, a first projection size 32 of the first conjugate pad 30 in a second direction L2 is equal to a second projection size 42 of the second conjugate pad 40 in the second direction L2, and the second direction L2 is perpendicular to the first direction L1. This ensures that the amounts of the sprayed liquid of the respective complexes on the pads are the same. In this embodiment, the fentanyl antibodies 71 are spaced apart and bound to the conjugate gold 72.

The second conjugate pad 40 is made of glass fiber material. The first conjugate pad 30 is made of glass fiber material.

The second conjugate pad 40 is stacked with the first conjugate pad 30 and the reaction pad 50, allowing the specimen 2, 2a to smoothly flow from the sample pad 20 through the first conjugate pad 30, the second conjugate pad 40, the reaction pad 50 and the absorption pad 60.

Each of the gold conjugated trithiocyanuric acid complexes 80 is bound to one of the gold conjugated fentanyl antibody complexes 70 via a gold-sulfur bond. Therefore, one of the gold conjugated fentanyl antibody complex 70 may bind simultaneously to ones of the gold conjugated trithiocyanuric acid complexes 80 to enhance signal strength and sensitivity. The reaction pad 50 is a nitrocellulose membrane. The reaction pad 50 includes a test line region T and a control line region C sequentially from the second conjugate pad 40 toward the absorption pad 60. The test line region T includes a plurality of fentanyl antigens 91. The control line region C includes a plurality of secondary antibodies 92, and each of the plurality of secondary antibodies 92 is configured to bind with the fentanyl antibody 71 of one of the gold conjugated fentanyl antibody complexes 70.

In use, the specimen 2, 2a (e.g., saliva) is dropped onto the sample pad 20. The specimen 2, 2a flows from the sample pad 20 toward the absorption pad 60 through capillary action.

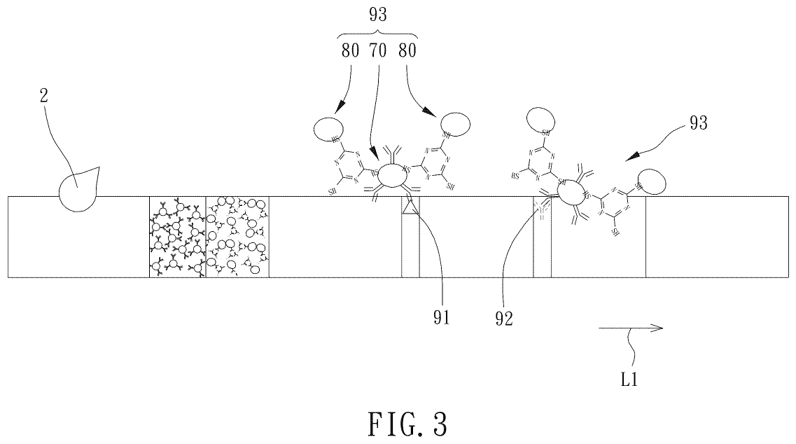

Please refer to FIG. 3. When the specimen 2 does not contain fentanyl, it flows through the first conjugate pad 30 to the second conjugate pad 40, enabling each gold conjugated fentanyl antibody complex 70 to bind with the plurality of gold conjugated trithiocyanuric acid complexes 80 to form a plurality of complexes 93. At least one complex 93 binds with at least one fentanyl antigen 91 in the test line region T, causing color development in the test line region T. At least one complex 93 also binds with at least one secondary antibody 92 in the control line region C, causing color development in the control line region C.

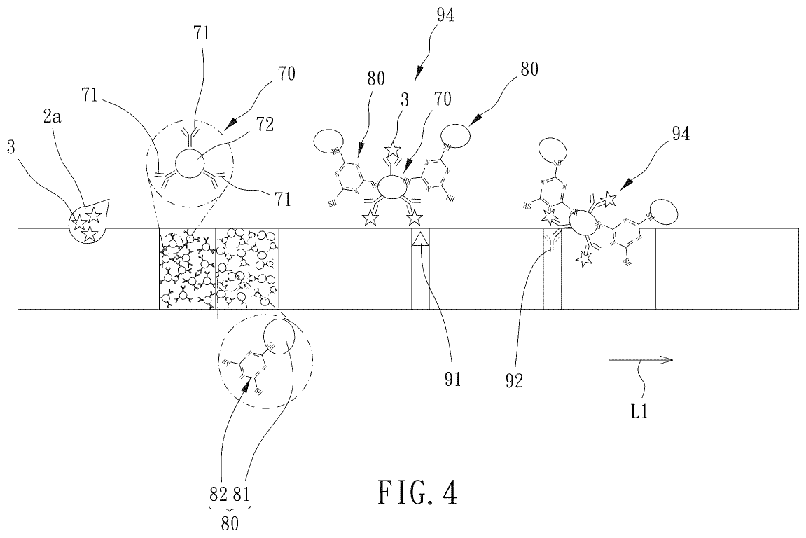

Please refer to FIG. 4. When the specimen 2a contains fentanyl 3, the fentanyl 3 binds with the fentanyl antibody 71 of the gold conjugated fentanyl antibody complexes 70 to form a plurality of fentanyl-gold conjugated fentanyl antibody complexes 94. Therefore, each of the fentanyl-gold conjugated fentanyl antibody complexes 94 cannot bind with at least one fentanyl antigen 91 of the test line region T and no color develops (disappearance of test line). Each of the fentanyl-gold conjugated fentanyl antibody complexes 94 binds with at least one secondary antibody 92 in the control line region C, causing color development.

Thus, using the same amount of antibody and antigen, the test line region T produces 2 to 3 times the conjugate gold signal intensity, amplifying the result difference between negative and positive and reducing false negatives and false positives. Therefore, this system has high sensitivity and can detect low concentrations of fentanyl 3. Specifically, the detection threshold of fentanyl 3 is 10 ng/mL, while commercially available devices have a threshold of 20 ng/mL. Hence, this invention can detect very low concentrations of fentanyl 3 with high sensitivity and strong detection signal.

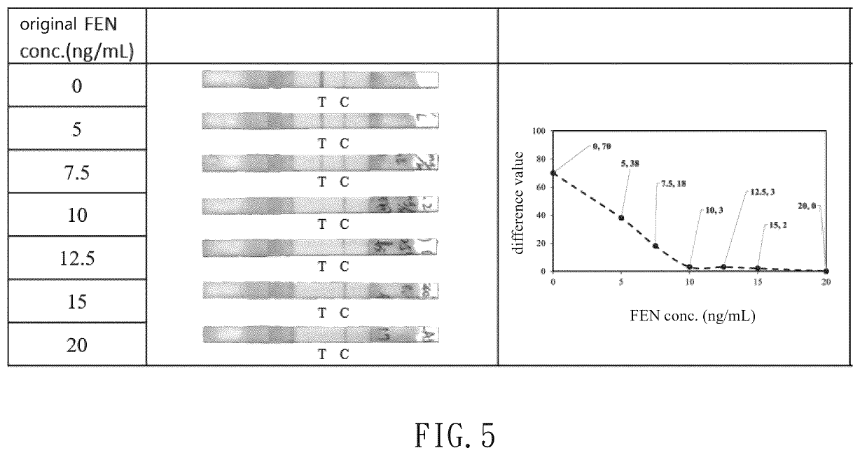

As shown in FIG. 5, the test results of the rapid test system for detecting fentanyl concentration of the present invention are presented. The experiment uses competitive immunoassay for testing. Specifically, the concentration of fentanyl (shown as original FEN conc. in the figure) in the specimen (e.g., saliva) gradually increases (from 0 ng/mL to 20 ng/mL), and the color of the test line region T on the rapid test system gradually fades. As a result, the difference between positive and negative is clear, and misjudgment is unlikely. Specifically, the detection threshold for the specimen is 10 ng/mL. Furthermore, based on the graph of the concentration of fentanyl in the specimen (original FEN conc.) and the difference value (shown on the right side of the chart in FIG. 5), the difference value increases with concentration. Therefore, the rapid test system 1 for detecting fentanyl concentration of the present invention produces a high-intensity detection signal.

Although particular embodiments of the invention have been described in detail for purposes of illustration, various modifications and enhancements may be made without departing from the spirit and scope of the invention. Accordingly, the invention is not to be limited except as by the appended claims.

What is claimed is:

1. A rapid test system for detecting fentanyl concentration including a base layer, a sample pad, a first conjugate pad, a second conjugate pad, a reaction pad and an absorption pad, wherein the sample pad, the first conjugate pad, the second conjugate pad, the reaction pad and the absorption pad are sequentially arranged above the base layer along a first direction, the first conjugate pad includes a plurality of gold conjugated fentanyl antibody complexes, the second conjugate pad includes a plurality of gold conjugated trithiocyanuric acid complexes, and the plurality of gold conjugated fentanyl antibody complexes are configured to bind with fentanyl.

2. The rapid test system for detecting fentanyl according to claim 1, wherein the plurality of gold conjugated fentanyl antibody complexes are disposed by spraying to form a first coating layer covering the first conjugate pad.

3. The rapid test system for detecting fentanyl concentration according to claim 2, wherein the plurality of gold conjugated trithiocyanuric acid complexes are formed by spraying and disposed over a second coating layer of the second conjugate pad.

4. The rapid test system for detecting fentanyl concentration according to claim 1, wherein the second conjugate pad is made of glass fiber material.

5. The rapid test system for detecting fentanyl concentration according to claim 1, wherein an amount of a sprayed liquid of the plurality of gold conjugated fentanyl antibody complexes on the first conjugate pad is equal to an amount of a sprayed liquid of the plurality of gold conjugated trithiocyanuric acid complexes on the second conjugate pad.

6. The rapid test system for detecting fentanyl concentration according to claim 1, wherein the second conjugate pad is stacked with the first conjugate pad and the reaction pad.

7. The rapid test system for detecting fentanyl concentration according to claim 1, wherein each of the gold conjugated trithiocyanuric acid complexes is bound to one of the plurality of gold conjugated fentanyl antibody complexes via a gold-sulfur bond.

8. The rapid test system for detecting fentanyl concentration according to claim 1, wherein the reaction pad includes a test line region and a control line region from the second conjugate pad toward the absorption pad, and the test line region includes a plurality of fentanyl antigens.

9. The rapid test system for detecting fentanyl concentration according to claim 8, wherein the control line region includes a plurality of secondary antibodies, each of the plurality of secondary antibodies is configured to bind with the fentanyl antibody of one of the plurality of gold conjugated fentanyl antibody complexes.

10. The rapid test system for detecting fentanyl concentration according to claim 3, wherein the second conjugate pad is made of glass fiber material; the first conjugate pad is made of glass fiber material; an amount of a sprayed liquid of the plurality of gold conjugated fentanyl antibody complexes on the first conjugate pad is equal to an amount of a sprayed liquid of the plurality of gold conjugated trithiocyanuric acid complexes on the second conjugate pad; the second conjugate pad is stacked with the first conjugate pad and the reaction pad; each of the gold conjugated trithiocyanuric acid complexes is bound to one of the plurality of gold conjugated fentanyl antibody complexes via a gold-sulfur bond; the reaction pad is a nitrocellulose membrane; the reaction pad includes a test line region and a control line region from the second conjugate pad toward the absorption pad, and the test line region includes a plurality of fentanyl antigens; the control line region includes a plurality of secondary antibodies, each of the plurality of secondary antibodies is configured to bind with the fentanyl antibody of one of the plurality of gold conjugated fentanyl antibody complexes; a first projection size of the first conjugate pad in a second direction is equal to a second projection size of the second conjugate pad in the second direction, and the second direction is perpendicular to the first direction.
